(19) United States
(12) Patent Application Publication
(10) Pub. No.: US 2023/0337658 A1
(43) Pub. Date: Oct. 26, 2023

(54) COMPOSITIONS AND METHODS FOR ENHANCING SPERM CELL QUALITY

(71) Applicant: ABS Global, Inc., DeForest, WI (US)

(72) Inventors: Elon Roti-Roti, Deerfield, WI (US); Xiao Peng, Middleton, WI (US); Rebecca Winters, DeForest, WI (US); Ashley Schroeder, Beaver Dam, WI (US)

(21) Appl. No.: 17/877,385

(22) Filed: Jul. 29, 2022

Related U.S. Application Data
(60) Provisional application No. 63/334,636, filed on Apr. 25, 2022.

(51) Int. Cl. A01N 1/02; C12N 5/076

(52) U.S. Cl. CPC A01N 1/0226; C12N 5/061; C12N 2501/999

(57) ABSTRACT

The present disclosure relates generally to potassium channel blocker compounds and compositions thereof; and methods for improving reproductive cell viability and quality, during and/or after one or more of staining, freezing, thawing, cell sample enrichment, packaging, or in vitro fertilization using the potassium channel blocker compounds.

### CROSS-REFERENCE TO RELATED APPLICATIONS

This application claims the benefit of and priority to U.S. Provisional Appl. No. 63/334,636 filed on Apr. 25, 2022, which is incorporated herein by reference in its entirety for any and all purposes.

### TECHNICAL FIELD

The present technology relates to the field of animal husbandry and breeding. In particular, the present disclosure includes improved compositions and methods for use with reproductive cell samples, and in fertilization processes. Such compositions and methods provide improved viability, enhanced protection and positive functional improvement of reproductive cells.

### BACKGROUND

An important aspect of animal husbandry, particularly in agriculture, is the collection and use of reproductive cells, e.g., sperm cells (spermatozoa), oocytes, zygotes, embryos, embryonic stem cells and spermatogonial stem cells. Generally, sperm cells are collected in the form of raw ejaculate from male animals. Subsequent use and manipulation of the sperm cells requires that the viability and function of the cells be maintained for hours or even days.

Artificial insemination (AI) and in vitro fertilization (IVF) are common techniques used in cattle and swine farming. Generally, semen samples need to be sorted and/or preserved prior to long-term storage. The sexing process subjects the sperm to cellular insults (Alvarz and Storey, 1992). These stresses decrease the viable cell population, and rapid losses are expected during at least three steps: incubation (at about 19° C.) before staining and sexing; the staining step; and during freezing for long-term storage. For example, stress during sexing can cause premature capacitation-like changes that minimize the sperm cell's ability to undergo timely capacitation in the reproductive tract, limiting fertilization potential (Leahy, T. et al., Reproduction 2011, 142, 759-778; de Graaf, S. P. et al., Bioscientifica Proceedings 2014, DOI:10.1530/biosciprocs.8.035). Induced oxidative DNA damage in sperm decreases fertilization rates and high levels of damage cause developmental arrest after embryonic transcript activation (Aitken et al., 2009; Fatehi et al., 2006). Various types of mediums, extender solutions and compounds have been developed to reduce the metabolic activity of sperm and allow for extended preservation. However, commercially available media do not provide the necessary performance characteristics, and may not allow for adequate maintenance of viability and/or activity of sperm. Additionally, commercially available media also can cause interference with downstream use of the sperm, such as during sex-sorting or in IVF or AI. Therefore, new and improved compounds, media and/or extender solutions are needed to improve AI, IVF and embryo culture outcomes.

### SUMMARY

Certain aspects and embodiments of the claimed invention are summarized below. These embodiments are not intended to limit the scope of the claimed invention, but rather serve as brief descriptions of possible forms of the claimed invention. The claimed invention may encompass a variety of forms which differ from these summaries.

One aspect of the disclosure relates to compositions comprising a reproductive cell selected from the group consisting of a sperm cell, an oocyte, an embryo, an embryonic stem cell and a spermatogonial stem cell; and an effective amount of a potassium channel blocker compound for enhancing cell viability during and/or after one or more of storage, staining, freezing, thawing, cell sample enrichment, packaging, or in vitro fertilization.

In any embodiments of the compositions, the potassium channel blocker compound comprises clofilium or a salt thereof (collectively referred to as ‘clofilium’). In any embodiments the potassium channel blocker compound comprises clofilium tosylate. In any embodiments the potassium channel blocker compound is present at a concentration ranging from 1 μM to 100 μM.

In any embodiments, the media formulation (i.e., the medium) comprises an additive. In yet other embodiments, the additive is selected from the group consisting of antioxidants, phosphatidylserine (PS), coumarin compounds or pyranocoumarin compounds, zinc chloride, coenzyme Q10, a nonsteroidal anti-inflammatory drug (NSAID), linolenic acid, fatty acids, D-aspartic acid, and combinations thereof. In any embodiments, the medium includes sodium fluoride.

In any embodiments, the composition comprises a plurality of sperm cells. In any embodiments, the sperm cells comprise mammalian sperm cells. In any embodiments, the mammalian sperm cells comprise human, bovine, porcine, equine, ovine, elk, or bison sperm cells.

In any embodiments, the composition comprises a medium. In any embodiments, the medium comprises one or more of a cryoprotectant, a buffer, a diluent, an energy source, an extender medium, and an antibiotic.

One aspect of the disclosure relates to a media composition comprising an amount of a potassium channel blocker compound effective to improve the function of sperm, oocyte, embryo, embryonic stem cell or spermatogonial stem cell, wherein the improvement in function comprises improvement in one or more of sexed semen production, efficiency of the sexing process, fertility/viability/physiological function of sexed semen, in vitro fertilization, rates of embryo production and/or increased implantation, and live births.

Another aspect of the disclosure relates to a composition comprising semen, an extender composition, and an effective amount of a potassium channel blocker compound, wherein the semen provides a concentration of motile sperm in the composition ranging from about 0.01 M motile sperm/mL to about 2000 M motile sperm/mL.

One aspect of the disclosure relates to methods for enhancing a reproductive cell viability during and/or after one or more of staining, freezing, thawing, cell sample enrichment, packaging, or in vitro fertilization. The methods may include adding to the reproductive cells, an effective amount of a potassium channel blocker compound or a composition thereof, wherein the potassium channel blocker compound is added at a concentration ranging from 1 nM to 100 mM.

Another aspect of the disclosure relates to methods of protecting sperm cells throughout the sexing process comprising adding to the sperm cells an effective amount of a potassium channel blocker compound or a composition thereof, wherein the potassium channel blocker compound is added before, during and/or after the sexing process at a concentration ranging from 10 nM to about 100 mM. In some embodiments, the potassium channel blocker compound is added before, during and/or after the sexing process at a concentration ranging from about 10 nM to about 100 μM. In some embodiments, the potassium channel blocker compound is added before, during and/or after the sexing process at a concentration ranging from about 1 mM to about 50 mM.

One aspect of the disclosure relates to methods of eliciting a positive functional improvement in sperm cells at the post-thaw stage, the methods comprising, adding to the sperm cells at a pre-freeze, an effective amount of a potassium channel blocker compound or a composition thereof, wherein the potassium channel blocker compound is added at a concentration ranging from about 0.001 μM to about 50 mM.

Another aspect of the disclosure relates to methods of improving quality of a semen sample comprising contacting the semen sample with an effective amount of a potassium channel blocker compound or a composition thereof, wherein the potassium channel blocker compound is added at a concentration ranging from about 0.001 μM to about 50 mM.

In any embodiments of any of the methods described herein, the potassium channel blocker compound is clofilium tosylate. In any embodiments of the methods described herein, the reproductive cell is selected from the group consisting of a sperm cell, an oocyte, an embryo, an embryonic stem cell, and a spermatogonial stem cell. In yet other embodiments of the methods described herein, the reproductive cells comprise sperm cells. In any embodiments of the methods described herein, the sperm in cells comprise mammalian sperm cells. In other embodiments of the methods described herein, the mammalian sperm cells comprises human, bovine, porcine, equine, ovine, elk, or bison sperm cells.

In any embodiments of the methods described herein, the potassium channel blocker compound is included in a medium. In any embodiments of the methods described herein, the medium comprises one or more of a cryoprotectant, a buffer, a diluent, an energy source, an extender medium, and an antibiotic. In other embodiments of the methods described herein, the medium comprises an extender medium. In any embodiments of the methods described herein, the methods further include adding the potassium channel blocker to the reproductive cells or sperm cells at least one time to maintain an effective amount of the potassium channel blocker. In any embodiments of the methods described herein, the methods further include adding the potassium channel blocker to the reproductive cells or sperm cells two or more times at different points in the methods to maintain an effective amount of the potassium channel blocker. In any embodiments of the methods described herein, the methods further include adding the potassium channel blocker to the reproductive cells or sperm cells at the staining step. In any embodiments of the methods described herein, the methods further include adding the potassium channel blocker to the reproductive cells or sperm cells at the centrifugation step. In any embodiments of the methods described herein, the methods further include adding the potassium channel blocker to the reproductive cells or sperm cells prior to the cryopreservation step.

### BRIEF DESCRIPTION OF THE DRAWINGS

To illustrate the disclosure, depicted in the drawings are certain features of the aspects and embodiments of the disclosure. However, the disclosure is not limited to the precise arrangements and instrumentalities of the aspects depicted in the drawings.

FIG. 1 is a flowchart showing the addition of clofilium tosylate at various stages during the sexing process, each media that could contain clofilium tosylate is designated by a star.

FIGS. 2A and 2B show a comparison in normalized percent motile cells and curvilinear velocity (VCL), respectively, at 0 and 2 hr of post-thaw straw material maintained at 37° C. with the treatments, for Control, DMSO, and Clofidium (0.2 μM and 5 μM).

FIG. 3A is a plot showing the % cleaved embryos at Day 2 and FIG. 3B is a plot showing the % Blastocysts at Day 7, both using 5 μM clofilium tosylate normalized to control.

FIG. 4 is a plot showing percentage of eligible cells (live, sexed X chromosome bearing cells) using 5 μM clofilium tosylate compared to Control, at the start of the collection process and at the end of the collection process.

FIG. 5 shows the pre-freeze motile cell quantification for straws/catch tube using 5 μM clofilium tosylate compared to Control.

FIG. 6A shows the post-freeze motile cell quantification using 5 μM clofilium tosylate compared to Control and FIG. 6B shows the post-freeze motile cell quantification as a measure of the insemination dose difference between the two samples.

### DETAILED DESCRIPTION

It is to be appreciated that certain aspects, modes, embodiments, variations and features of the present methods are described below in various levels of detail in order to provide a substantial understanding of the present technology.

### Definitions

Throughout this application, various embodiments of the present technology may be presented in a range format. It should be understood that the description in range format is merely for convenience and brevity and should not be construed as an inflexible limitation on the scope of the present technology. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 10% (including, e.g., 1%, 5% or 10%) in either direction (greater than or less than) of the number unless otherwise stated or otherwise evident from the context (except where such number would be less than 0% or exceed 100% of a possible value).

The expression “comprising” means “including, but not limited to.” For example, compositions and methods include the recited elements, but do not exclude others. “Consisting essentially of” shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention. It will be understood that use of any of these expressions—“comprising,” “consisting essentially thereof,” or “consisting of”—also contemplate and provides disclosure for use of any of the other terms.

As used herein, the terms “individual”, “patient”, or “subject” can be an individual organism, a vertebrate, or a mammal. In some embodiments, the individual, patient or subject is cattle, pig, or sheep.

As used herein, the term “effective amount” refers to an amount sufficient to produce a biological effect.

As used herein, “fertility” includes one or more of the following: a level or degree of ability of an animal to conceive and bear young; a level or degree of ability of an animal to become pregnant; a level or degree of ability of an animal to reproduce; a level or degree of ability of a spermatozoa to fertilize an oocyte; a level or degree of ability of an oocyte to be fertilized by a spermatozoa; and a level or degree of ability of an oocyte fertilized by a spermatozoa to develop into a zygote capable of progressing through embryonic and fetal development. Various methods of evaluating fertility, such as in gametes, are described in U.S. Patent Pub. 2020/0347347 by Roti-Roti, which is incorporated herein by reference and for all purposes.

The term “sexing” or “sex selection” as used herein refers to any process that selects X-chromosome bearing or Y-chromosome bearing sperm in cells from a population that comprises a mixture of both X-chromosome and Y-chromosome bearing sperm cells. The sperm in cell population can be raw ejaculate, or any other mixture or sperm cells. The sexing process can be accomplished using a number of different techniques, including droplet sorting, mechanical sorting, and laser ablation.

As used herein, the terms “enrichment” or “cell sample enrichment” may comprise sorting, separating, removing, or otherwise acting on a subset of cells in a sample, so as to provide for a sample comprising a fewer number of cells with a first set of one or more characteristics and a greater number of cells with a second set of one or more characteristics.

The term “reproductive cell” as referred herein is defined as sperm, eggs, and the formation of embryo/blastocyst, also gametes; haploid cells; germ cells; sex cells; sperm cells and egg cells.

The term “medium” or “media” as used herein refers to an essentially liquid composition that may contain nutrients, salts, and other substances or constituents.

The term “ejaculate” as used herein refers to the combination of semen and spermatozoa, which may comprise any amount of seminal plasma, produced by a male mammal, as released by ejaculation.

The term “seminal fluid components” as referred herein is the substances that make up and/or are commonly found in mammalian semen. Seminal fluid components include, but are not limited to, amino acids, prostate specific antigen, proteolytic enzymes, citric acid, citrate, sialic acid, vitamin C, acid phosphatase, fibrinolysin, lipids, fructose, prostaglandins, phosphorylcholine, glycerophosphocholine, flavins, basic amines such as putrescine, spermine, spermidine and cadaverine, zinc, galactose, mucus and other organic and inorganic constituents.

The collection and use of sperm cells is a central part of animal husbandry and breeding. Sperm cells are collected in the form of raw ejaculate from male animals and must be stored before further use. Storage can comprise hours or even days. Additionally, cell samples are usually manipulated in one or more ways before use. Thus, it is important to maintain the viability and function of the cells throughout the process.

Sexing procedures disclosed can be implemented for use with fresh, un-extended ejaculate. However, there are inherent issues to using fresh ejaculate that is not supplemented with an extender, as an ejaculate declines in quality continually after collection. Sperm that undergoes sexing is exposed to numerous insults including temperature swings, high dilution, and pH changes during cell processing. Insults during sexing include shear stress, high fluid pressure, and the high force caused by the sexing process on cytometers (Gamer and Seidel, 2003; Garner, 2006). These insults lead to a decrease in the number of cells recovered after processing.

That loss of cells during processing, while detrimental, is not the main source of projected product loss. The major loss is due to fresh ejaculates having a steady increase in dead cell population over time after collection. A major factor of this is that the fresh ejaculates must be stored at close to room temperature, as the sexing process happens at room temperature. Keeping the ejaculate at room temperature, rather than at a cooler temperature that better preserves cell survivability prevents time spent on equilibrations to the cooler temperature and allows sexing of the ejaculates continuously. This also increases the rapidity in which the viable cells are lost before the sexing process begins. This continual loss of viability when paired with the time required to perform the sexing and the packaging steps led to the standard policy of freezing a sexed ejaculate no later than 12 hours after collection.

This policy of having to package an ejaculate no later than 12 hours after collection leaves a lot of ejaculate volume behind due to surplus ejaculate volume. These excess volumes cannot be utilized because fresher ejaculates are being collected before they can be used entirely. Ejaculates are collected every 6 hours, 24 hours a day, to prevent a lapse in instrument running time due to lack of sample. This means that ejaculates from an early morning collection frequently still have viable volume left to run when the second daily collection arrives but are still removed and replaced in favor of the fresher ejaculate because the older sample is not likely to run an additional 6 hours on the instrument. Favoring fresh ejaculates leads to a second major source of loss: downtime on sexing instruments. All ejaculates from a given time point are removed and replaced at the same times each day, meaning instruments are shut down, cleaned, and restarted four times daily. This means that a minimum of 40 minutes of run time is lost, four times daily, for each instrument. These numbers calculated in a number of cells is 46.9×10⁸ skewed cells uncollected per instrument per day; which translates to approximately one thousand insemination doses lost per day.

An extender or other medium formulated for use in a sperm sexing facility could help to mitigate losses. For example, the medium can slow the decline of sperm cells held at room temperature before sexing, allowing a larger number of ejaculates to be collected at a single time point, and decreasing the number of times per day ejaculates are collected. In this model, instruments would only be shut down to change to a new ejaculate as needed on a bull by bull basis, rather than the entire production floor at once. This would decrease the time it takes to change to a new bull, as it increases the available staff per instrument. Maintaining cell viability also means ejaculates could be run until exhaustion, maximizing the number of sexed sperm obtained per ejaculate, and decreasing the total number of times per day a bull change would be performed per instrument. By mitigating these causes of cell loss, the number of insemination doses produced would increase making the superior product more available to farmers globally.

A potassium channel blocker compound would also be useful in reducing or mitigating cell stress involved in a staining step or process, such as a staining process with Hoechst 33342 prior to enriching or sexing a cell population on an instrument. For example, in a staining process, a semen sample may be mixed with staining media and other compounds which alter the concentration and pH of the semen sample, and the semen sample may be subjected to elevated temperatures, such as temperatures above 30° C. The changes in sample chemistry, concentration, and temperature are stressors on the cells that impact cell health. Cell viability is improved by reducing the stressors, or by reducing or mitigating the impact of the sources of stress on the cells. A potassium channel blocker compound is used to reduce or mitigate the impact of the stressors on the cells thereby maintaining cell viability, reducing cell loss, and maximizing the number of sexed sperm that may be obtained per ejaculate.

In addition, a large amount of cell loss is observed during the freeze-thaw process. Both conventional (i.e. non-sexed) and sexed semen are typically frozen in straws for storage and distribution. The straws must be subsequently thawed prior to use for insemination or fertilization. This process of freezing and thawing results in the death of a large proportion of the cells in the straw. The compositions and processes disclosed herein can be used to alleviate this cell loss due to the freeze-thaw process.

### Compositions

The present disclosure relates to certain potassium channel blocker compounds, and compositions thereof, and methods that improve reproductive cell viability and activity.

In some embodiments, the potassium channel blocker compound is an element of a pre-made or pre-formulated media (or a composition). In some embodiments, the potassium channel blocker compound is added (e.g., to the reproductive cells) on its own as a discrete element or step in a process described herein.

In some embodiments, the potassium channel blocker compounds, compositions and/or methods described herein may be used in a conventional process (e.g., conventional (i.e. non-sexed) semen). For example, the potassium channel blocker compounds (and/or compositions) described herein may be added to conventional semen after it has been collected. In some embodiments, the potassium channel blocker compounds (and/or compositions described) herein are added to conventional semen immediately prior to packaging in straws. In some embodiments, the potassium channel blocker compounds (and/or compositions described) herein are added to conventional semen immediately before usage in an AI or IVF process.

In some embodiments, the potassium channel blocker compounds, compositions and/or methods described herein may be used in a sexing process (e.g., sexed semen). In some embodiments, the potassium channel blocker compounds (and/or compositions) described herein may be added to semen that is to be sexed. In some embodiments, the potassium channel blocker compounds (and/or compositions described) herein are added to semen that is to be sexed at one or more of the following stages, including 1) immediately after collection; 2) during incubation or pre-instrument QC (quality control); 3) immediately prior to, during, or after staining; 3) pre-sexing instrument; 4) post-instrument in the catch tube; 5) prior to centrifugation; 6) after centrifugation; 7) pre-packaging, either prior or post-cryopreservation addition; and 8) post-thaw, such as before an AI or IVF event.

In some embodiments, the present specification provides and includes potassium channel blocker compounds and/or compositions that impart increased activity and viability to reproductive cells, in particular, sperm cells. The present specification also provides and includes methods for processing reproductive cell samples, wherein the methods and processes produce samples in which the sperm cells have increased viability and activity, and improve production efficiency. The present specification, also provides and includes the reproductive cell samples produced by these methods, wherein reproductive cells in the samples have increased viability and activity. The present specification still further provides and includes methods using these reproductive cell samples with increased viability and activity, including sexing (selecting X-chromosome bearing or Y-chromosome bearing cells), sorting, separating, freezing, artificial insemination, in vitro fertilization, cooling and transport, and related processes.

In various aspects and embodiments, the present disclosure relates to new compositions, new media formulations, new processes for addition of the potassium channel blocker compound (e.g., clofilium or a salt thereof (e.g., clofilium tosylate)), to sperm cells (before/during/after sexing, or in a conventional process without sexing) and/or to embryos (before/during/after IVF). In some embodiments, the potassium channel blocker compounds described herein are included in a composition or a media before adding to sperm cells and/or to embryos. In some embodiments, the potassium channel blocker compounds described herein are added directly (e.g., not included in a composition or a media before use) to sperm cells and/or to embryos. In embodiments, the present disclosure relates to improvements in conventional (i.e. non-sexed) semen production. In embodiments, the present disclosure relates to improvements in sexed semen production. Exemplary improvements include but are not limited to, improved efficiency of the sexing process and improved fertility/viability/physiological function of sexed semen; improvements in in vitro fertilization, including improved rates of embryo production and/or increased implantation, live births.

In some embodiments, provided herein are compositions which include a reproductive cell selected from the group consisting of a sperm cell, oocyte, embryo, embryonic stem cell and a spermatogonial stem cell; an effective amount of a potassium channel blocker compound for enhancing cell viability during and/or after one or more of staining, freezing, thawing, cell sample enrichment, packaging, or in vitro fertilization; and optionally, a medium. In some embodiments, the medium is an extender medium. In other embodiments, the medium may be a maturation medium or other medium used in an IVF process, or could be a staining solution in a sexing process, or a collection medium (e.g., comprising a cryoprotectant) in the sexing and/or the conventional process.

As used herein, a potassium channel blocker may be a channel blocker (e.g., a pore blocker) or a channel inhibitor. Suitable potassium channel blocker compounds are known in the art and include compounds known to block Slo3 channels, such as for example, clofilium, quinine, quinidine mibefradil, 4-aminopyridine, tetraethylammonium, barium, charybdotoxin, progesterone, or a salt thereof. In some embodiments, the potassium channel blocker compound is clofilium (N-[4-(4-chlorophenyl)butyl]-N,N-diethylheptan-1-aminium), which has the following stricture:

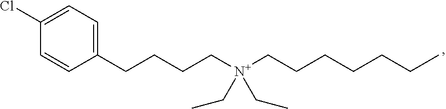 [SMILES: CCCCCCC[N+](CC)(CC)CCCCc1ccc(Cl)cc1]

or a salt thereof

In some embodiments, the potassium channel blocker compound is clofilium tosylate, which is a small molecule antagonist of the Slo3 K⁺ channel.

The potassium channel blocker compound (e.g., clofilium tosylate) can be provided in an amount effective to enhance cell viability during and/or after one or more of staining, freezing, thawing, cell sample enrichment, packaging, or in vitro fertilization. Depending on the type, the potassium channel blocker compound can be provided between about 0.1 nM and about 100 mM, including, for example, about 0.001 μM to about 50 mM, about 0.01 μM to about 500 μM, about 0.1 μM to about 250 μM, about 1 μM to about 100 μM, about 2 μM to about 50 μM, about 3 μM to about 30 μM, about 4 μM to about 20 μM, about 5 μM to about 10 μM, or about 5 μM. In some embodiments, the potassium channel blocker compound is present at a concentration ranging from about 1 μM to about 100 μM. In some embodiments, the potassium channel blocker compound is present at a concentration ranging from about 5 μM to about 50 μM. In some embodiments, the potassium channel blocker compound is present at a concentration of less than about 500 μM, or alternatively less than about 100 μM, or alternatively about less than 50 μM, or alternatively less than about 30 μM, or alternatively less than about 20 μM, or alternatively less than about 10 μM, or alternatively less than about 5 μM, or alternatively less than about 3 μM, or alternatively less than about 2 μM In some embodiments, the potassium channel blocker compound is present at a concentration of greater than about 0.01 μM or alternatively greater than about 0.1 μM, or alternatively about greater than 1 μM, or alternatively greater than about 2 μM, or alternatively greater than about 5 μM, or alternatively greater than about 10 μM, or alternatively greater than about 15 μM, or alternatively greater than about 20 μM, or alternatively greater than about 50 μM. In some embodiments, the potassium channel blocker compound is present at a concentration ranging from about 10 nM to about 100 μM. In some embodiments, the potassium channel blocker compound is present at a concentration ranging from about 1 mM to about 50 mM. In some embodiments, the potassium channel blocker compound is present at a concentration ranging from about 5 μM to about 50 μM. In some embodiments, the potassium channel blocker compound is clofilium tosylate, and it is present at a concentration of about 10 nM to about 100 μM. In some embodiments, the potassium channel blocker compound is clofilium tosylate, and it is present at a concentration of less than about 5 μM. In some embodiments, additional potassium channel blocker is tetraethylammonium or barium compound, and it is present at a concentration of about 1 mM to about 50 mM. In some embodiments, the potassium channel blocker compounds described herein (e.g., clofilium tosylate) are included in a composition or a media before adding to sperm cells and/or to embryos, and the concentrations described herein are the concentration of the potassium channel blocker compounds in the composition or media. In some embodiments, the potassium channel blocker compounds described herein (e.g., clofilium tosylate) are added directly (e.g., not included in a composition or a media before use) to sperm cells and/or to embryos, and the concentrations described herein are the concentration of the potassium channel blocker compounds in the resulting mixture (resulting composition) after addition.

In some embodiments, the potassium channel blocker compound (including but not limited to clofilium) is provided in an amount effective to enhance cell viability during and/or after one or more of staining, freezing, thawing, cell sample enrichment, packaging, or in vitro fertilization, together with one or more other fertility enhancing compounds. The one or more other fertility enhancing compounds may include an adenylyl cyclase inhibitor such as SQ 22536, an estrogen receptor β agonist such as (R)-DPN, a c-Jun kinase inhibitor such as BI-78D3, a Bax channel inhibitor such as Bax, and/or a cyclin dependent kinase 1 inhibitor such as RO 3306. Effective amounts for these compounds may range from about 0.1 uM to about 30 uM. One or more of the compounds may be used in addition to clofilium to further improve the fertility of the sample.

In addition to the potassium channel blocker, the compositions may also include one or more types of reproductive cells. Suitable reproductive cells, include, without limitation, sperm cells, oocytes, embryos, embryonic stem cells and spermatogonial stem cells. In some embodiments, the composition may include a single sperm cell or a plurality of sperm cells. In some embodiments, the composition may include a single oocyte or a plurality of oocytes. In some embodiments, the composition may include a single embryo or a plurality of embryos. In some embodiments, the composition may include a single embryonic stem cell or a plurality of embryonic stem cells. In some embodiments, the composition may include a single spermatogonial stem cell or a plurality of spermatogonial stem cells. In some embodiments, the composition may include a plurality of sperm cells. In some embodiments, the sperm cells include mammalian sperm cells. Suitable mammalian sperm cells may include, without limitation, human, bovine, porcine, equine, ovine, elk, or bison sperm cells. The concentration of reproductive cells in the composition can be adapted based on the type of cells and the process stage. For example, traditionally, sex selection is performed at 66 million cells per mL (M/mL). Accordingly, the initial cell concentration can be higher (e.g., 1000 M/mL or less), which can then diluted in a medium or buffer to a concentration of 66 M/mL for sexing.

Different sexes of livestock are preferred depending on the application. For example, only female dairy cattle produce milk, and male cattle have greater muscle mass for beef production. Therefore, it is desirable to select sperm cells based on their chromosomal content: X-chromosome bearing sperm to produce female offspring and Y-chromosome bearing sperm to produce male offspring. Semen suitable for use in the present teachings can be semen from any type of mammalian livestock, including, such as but without limitation, bovine semen, porcine semen, ovine semen, or equine semen. Semen may also be from, for example, a ruminant animal, an even-toed ungulate animal, or an odd-toed ungulate animal. Bovine semen, such as Bos taurus semen or Bos indicus semen and porcine semen, such as Sus scrofa semen, are especially preferred. Semen suitable for use in the present teachings can be semen from a collected ejaculate or epididymal semen. Methods of collecting both types of semen are known in the art.

The composition of the present technology may further include a medium. Suitable mediums, include without limitation, an extender medium, a cryoprotectant, a buffer, a diluent, an energy source, an extender medium, an antibiotic, and a bolus. The potassium channel blocker may be added to the composition separately or included in the medium. In some embodiments, the potassium channel blocker, e.g., clofilium or a salt thereof, is included in the extender medium. In some embodiments, the potassium channel blocker can be added altogether in a single dose or as a bolus, slowly titrating in, and combinations of the two. In any embodiment, the media composition contains the potassium chain blocker compound, and is combined with cells in a variety of ways, for example by using a set volume of media; a set ratio of media to cell sample, or media provided at a set volume in relation to a measured aspect of the sample (i.e., sperm cell concentration). In certain embodiments, media is added to the sample, in other embodiments, the sample is added to the media. In other embodiments, both sample and media are added to a third container/receptacle.

The present technology provides potassium channel blocker compounds and compositions thereof. In some embodiments, the potassium channel blocker compound includes clofilium or a salt thereof (e.g., clofilium tosylate). The potassium channel blocker is present, e.g., in the composition and/or medium in an amount effective to improve the function of sperm, oocyte, embryo, embryonic stem cell or spermatogonial stem cell, wherein the improvement in function comprises improvement in semen production (sexed semen production and/or non-sexed semen production), improvement in efficiency of the sexing process, improvement in fertility/viability/physiological function of semen (sexed semen and/or non-sexed semen), improvement in in vitro fertilization, improvement in rates of embryo production and/or increased implantation, and live births.

The present technology preserves the viable, motile sperm population eligible for sexing and significantly reduce the cost of sexed semen to farmers by increasing yield, such as by increasing the number or percentage of successful or viable pregnancies from artificial insemination events using said semen. The ideal extender formulation must maintain a high motile sperm population and must not interfere with the ability to separate the X and Y populations using a fluorescent DNA stain, which is required to separate the two cell populations on the sexing cytometry instruments.

In some embodiments, additives may be included in a composition (and/or a medium) comprising the potassium channel blocker compounds to bring about the desired properties and create the inventive media. In any embodiment, the compositions comprise a medium which includes at least one additive selected from the group consisting of antioxidants, phosphatidylserine (PS), coumarin compounds or pyranocoumarin compounds, zinc chloride, coenzyme Q10, a nonsteroidal anti-inflammatory drug (NSAID), linolenic acid, fatty acids, D-aspartic acid, and combinations thereof. In some embodiments, the medium contains sodium fluoride. In any embodiment, the additives comprise one or more of phosphatidylserine (PS), decursin, zinc chloride, coenzyme Q10, acetylsalicylic acid (aspirin), linolenic acid, fatty acids, D-aspartic acid, sodium fluoride, and combinations thereof. in some embodiments, all of the additives are included in the formulation. In other embodiments, one or more of decursin, zinc chloride, coenzyme Q10, acetylsalicylic acid (aspirin), linolenic acid, fatty acids, D-aspartic acid, or sodium fluoride are omitted. The additives, when present, may be included in a concentration range of from about 0 to about 100 mM or 0 to 1000 μg/mL.

In any embodiment, the medium further includes a buffer. For applications where cell survivability and/or shelf life is of preeminent concern, the buffer may be TRIS or HEPES. In certain embodiments, TRIS may be used due to its longer shelf life in the formulation as measured by pH stability.

In other embodiments, the medium may be supplemented with a salt such as sodium chloride (NaCl), potassium chloride (KCl), calcium chloride dehydrate (CaC₁₂(H₂O)₂), magnesium chloride hexahydrate (MgC₁₂(H₂O)₆), sodium bicarbonate (NaHCO₃), sodium phosphate dihydrate (NaH₂PO₄(H₂O)₂), potassium phosphate (KH₂PO₄), and sodium fluoride (NaF).

In one embodiment, the medium may further include antioxidants, which are added to decrease the amount of stress the cells are subjected to during the sexing process. All sperm are exposed to UV light during the sexing process, which typically causes oxidative damage to DNA (rather than direct strand breaks), and sperm is likely subject to elevated reactive oxygen species (ROS) during the cryoprotectant step (Aitken et al., 2015; Farber, 1994). ROS can also cause DNA damage such as single and double strand breaks, and base pair modification (Richter et al., 1988). Fatehi et al. (2006) reported oocytes fertilized with DNA damaged bovine spermatozoa exhibited cleavage rates similar to controls, but further development halted in the damaged experimental group. The compositions of the present technology which include sperm cells treated with potassium channel blocker compound may exhibit less DNA damage than non-extended controls.

In some embodiments, the compositions comprise Nonsteroidal Anti-inflammatory Drugs (NSAIDs), which are a class of drugs and compounds capable of reducing inflammation, primarily through inhibition of cyclooxygenase enzymes (COX-1 and/or COX-2). The compositions can include one or more NSAID including, but not limited to: salicylates, including aspirin (acetylsalicylic acid), diflunisal (Dolobid); salicylic acid and other salicylates, and salsalate (Disalcid); Propionic acid derivatives, including Ibuprofen, Dexibuprofen, Naproxen, Fenoprofen, Ketoprofen, Dexketoprofen, Flurbiprofen, Oxaprozin, and Loxoprofen; acetic acid derivatives, including indomethacin, Tolmetin, Sulindac, Etodolac, Ketorolac, Diclofenac, Aceclofenac, and Nabumetone; enolic acid (Oxicam) derivatives, including Piroxicam, Meloxicam, tenoxicam, Droxicam, Lornoxicam, Isoxicam, and phenylbutazone (Bute); anthranilic acid derivatives (Fenamates), including mefenamic acid, meclofenamic acid, flufenamic acid, and tolfenamic acid; selective COX-2 inhibitors (Coxibs), including Celecoxib, Rofecoxib, Valdecoxib, Parecoxib, Lumiracoxib, Etoricoxib, and Firocoxib; Sulfonanilides, including Nimesulide; and other NSAIDs, including Clonixin, Licofelone, and H-harpagide (in Figwort or Devil's Claw). In some embodiments, the compositions comprise coumarin compounds or pyranocoumarin compounds. In certain embodiments, the coumarin compound or pyranocoumarin compound comprises decursin.

In one embodiment, the medium may further include one or more of antioxidants, phosphatidylserine (PS), phosphatidylcholine, coumarin compounds, pyranocoumarin compounds, zinc chloride, coenzyme Q10, a nonsteroidal anti-inflammatory drug (NSAID), linolenic acid, fatty acids, D-aspartic acid, sodium fluoride, decursin, acetylsalicylic acid (aspirin), a salt (e.g., sodium chloride, potassium chloride, calcium chloride dihydrate, magnesium chloride hexahydrate, sodium bicarbonate, sodium phosphate dihydrate, potassium phosphate, sodium fluoride), a buffer (HEPES, Tris, etc.), a sugar source (glucose, fructose, etc.), citric acid, pyruvate, ascorbic acid, glycerol or other cryoprotective agents, natural or synthetic ice blockers, BSA or other protein source, egg yolk, polyvinylalcohol, polyvinylpyrrolidone, and other polymers. In one embodiment, the medium may also include beads, such as magnetic (or other) beads coated with lectin, DNA-binding moieties or ubiquitin-targeted moieties. For example, lectin-beads can be used to remove the dead cells during the staining process.

In any embodiment, by using the present technology, reproductive cell activity of stored and/or manipulated samples can be maintained or even increased, by the measure of either motility, fertilization, or both. Fertilization can be measured by blastocyst formation. Concentration and progressive motility of the sample may be further evaluated using a computer assisted sperm analysis platform (CASA) such as an integrated visual optical system (“IVOS”).

In any embodiment, provided is a composition with semen, an extender composition, and an effective amount of a potassium channel blocker compound, wherein the semen provides a concentration of motile sperm in the composition ranging from about 0.01 M motile sperm/ml to about 2000 M motile sperm/ml. For example, concentration of motile sperm in the composition may range from about 75-200 M motile sperm cells per mL in the stain reaction; about 25-100 M motile sperm cells per mL as the samples are on-instrument; about 0.25-4 M motile sperm cells/mL at the post-instrument stage; about 10-600 M motile sperm cells/mL after centrifugation; about 5-300 M motile sperm cells/mL at cooling/pre-freeze stage after addition of Part 2 of a two-part extender (e.g., Tris B); and about 0.25-15 M motile/mL at freeze. In some embodiments, the clofilium is added to a composition having semen in a concentration range of 400-2,000 M motile sperm/mL for the extender described in US PGPUB 2020/0347347 (Roti-Roti et al.) and the same extender with an amount of citrate.

The present technology provide a number of important benefits: extends cell viability, with a reduced loss of progressively motile cells; does not interfere with Hoechst 33342 (or an alternative) staining and red dye viability counterstaining of the cells which is necessary for proper sexing on the cytometers; and does not negatively interfere with fertilization capacity or embryonic development.

The present technology relates to a potassium channel blocker compound, e.g., clofilium or a salt thereof, as well as a composition or a medium thereof, which maximizes recovery and packaging of functional, fertilization competent sperm. This can improve the flexibility and efficiency of production by exhausting ejaculates, optimizing bull changes, and decreasing the need for backup ejaculates. An additional advantage is an increase in motile cells recovered post-processing (e.g., sexing). When semen samples are processed using the inventive compositions and methods, increased activity is seen. Increased activity can be increased viability, increased motility or both. Moreover, use of the present technology allows for greater yields of semen samples during and after processing. Specifically, the inventors have discovered that the inclusion of clofilium tosylate at various stages (including various stages of sexing) improves fertility and processing. This was a surprising and unexpected finding. For example, it was observed that including a potassium channel blocker compound such as clofilium tosylate in the process (including the sexing process) improves fertility of semen (e.g., sexed semen), as assessed by embryo cleavage and blastocyst formation post-IVF. Remarkably, semen samples (e.g., sexed semen samples) treated with clofilium tosylate were found to exhibit increased cellular function, as measured both by mitochondrial membrane potential and motility. Increased cellular function can also be measured by ATP production, plasma membrane integrity, and other cell physiological readouts. In addition, the presence of clofilium tosylate during the process of staining sperm cells with Hoechst dye was found to reduce the time required to achieve staining saturation, increase eligibility (e.g., the number of cells eligible for sexing), the number of cells eligible for sexing, increase resolution (separation of X- and Y-chromosome cell peaks in a histogram derived from interrogation of sperm cells by an instrument), and sex skew, with the potential to positively impact batch yields.

The present technology may exhibit enhanced sperm cell survivability and motility compared to commercially available extenders: Andromed® (Minitube, Delavan, Wis. USA) and OptiXell (IMV technologies, Maple Grove, Minn. USA), and a previously described media formulation CEP2 (Verberckmoes et al., 2004). The present technology may extend the window of cell survivability before sexing, while still maintaining cells measured as live and motile after the sexing process. The present technology may result in an increase of the percent of blastocysts per oocyte. In some embodiments, the compositions may result in less cleavage. In some embodiments, the present technology may successfully maintain the motile, viable sperm population for an enhanced period of time, e.g., 24 hours, before sexing, and may result in frozen-thawed sexed semen that meets quality control standards with no increased risk for batch failure compared to current standard operating procedures. Use of the potassium channel blocker compound, as well as the compositions thereof, therefore, has the potential to increase utilization of the total ejaculate volume and concurrently increase the number of insemination doses produced per ejaculate, increasing the availability of sexed semen for fanners. In any embodiment, the present technology may maintain sperm in a fertilization competent state. Fertilization competence includes, but is not limited to, the capability of sperm cells exposed to compositions according to the present technology for producing pregnancies via artificial insemination, and fertilization, cleavage, and blastocyst conversion both in vitro and in vivo. The compositions of the present technology which include sperm cells treated with potassium channel blocker compound, may produce more blastocysts per oocyte compared to non-extended, paired controls. In some embodiments, the potassium channel blocker compound may produce enhanced zygote/blastocyst formation from germ cells (i.e., increased fertilization or increased activity of reproductive cells).

Other advantages realized by use of the present technology are a decreased need for multiple collections of ejaculates or moving of animals to the process site. Additionally, the present technology, due to its maintenance of viability and motility of reproductive cells, can allow for shipping of ejaculates for further processing (e.g., sexing). This eliminates the need to move and quarantine animals.

Sources of reproductive cell samples are typically from ejaculate, obtained by methods commonly known in the art. The ejaculate samples can be a single source or pooled. In some embodiments, in vitro produced or expanded sperm cell populations may be used. Samples are obtained from animals, preferably mammalian animals; more preferably livestock; samples are most preferably porcine or bovine.

In some embodiments, the media composition containing the potassium channel blocker compound is utilized as a “hold media” to store raw ejaculate and minimize loss of reproductive cell components. In further embodiments, the composition is utilized as a “hold media” to store isolated sperm cells after processing and before use in breeding procedures. This can also be referred to as an “extender media” since samples remain viable for longer when the inventive media is used. In other embodiments, the composition functions as a medium to use for processing of reproductive cell samples that are used for further processing (such as, e.g., sexing). For example, the composition may be utilized as a staining media, a sexing processing media, and/or a freezing media.

In certain embodiments, compositions comprising reproductive cells and the inventive “hold media” maintained acceptable viability and/or motility for hours; in particular embodiments, the extension was for 24 hours. In other embodiments, the inventive compositions maintained an acceptable level of live cells throughout cell processing; in particular embodiments, the percentage of dead cells were ≤25% throughout sexing duration of processing.

Samples can be combined with potassium channel blocker compound and/or the improved media in a variety of ways. The potassium channel blocker compound and/or media can be added directly after collecting the raw ejaculate sample, within a set amount of time after collecting the raw ejaculate; or the raw ejaculate can be collected directly into the media.

Sperm cells that are exposed to the potassium channel blocker compound (or the inventive compositions thereof) can exhibit enhanced motility, viability, and functionality (including the ability to fertilize ova) over time, compared to sperm exposed to existing commonly-used media. Thus, these potassium channel blocker compound (or the compositions thereof) can enhance yields in both conventional and sexed semen production. The potassium channel blocker compound (or the inventive compositions thereof) can maximize recovery and packaging of functional, fertilization competent sperm.

The present technology may provide beneficial effects for IVF outcomes, measured as cleavage and blastocyst conversion rates, which may be due, at least in part, to mitigation of DNA damage, increased ROS production, and/or capacitation-like changes caused by sexing. The potassium channel blocker compound (or the compositions thereof) of the present technology may also allow for increased run time for each ejaculate and thereby increase frozen semen (e.g., sexed semen) product per volume of ejaculate collected and decrease the cost of each insemination dose. This allows semen (e.g., sexed semen) products to be more widely available to farmers who would profit from the use of semen (e.g., sexed semen) on their cattle farms. Further the compositions are applicable not only to frozen sexed bovine semen, but could also have applications in extending the life of a fresh ejaculate in a setting where extended transport times are required or specifically for preservation of ejaculates of impaired quality.

### Methods

One aspect of the disclosure relates to methods of processing mammalian reproductive cells comprising the steps of providing a mammalian reproductive cells sample, processing the mammalian reproductive cells sample, and adding potassium channel blocker compound (or a media composition thereof) of the present technology. In any embodiments the potassium channel blocker compound is included in a media composition before adding to the mammalian reproductive cells sample. In any embodiments the potassium channel blocker compound is added to the mammalian reproductive cells sample by itself. In any embodiments, the potassium channel blocker compound is clofilium or a salt thereof, e.g., clofilium tosylate. In any embodiment, the media includes an additive. In any embodiment, the processing comprises at least one step selected from the group consisting of collecting a semen sample, sexing, sorting, separating, freezing, artificial insemination, in vitro fertilization, cooling, transport, and related processes. In any aspect or embodiment, the sexing is accomplished via droplet sorting, mechanical sorting, micro fluidic processing, microchip processing, jet and air processing, flow cytometry processing, and laser ablation. In any aspect or embodiment, the mammalian reproductive cells are obtained from a male mammal. In yet other embodiments, the male mammal is a bull or boar. In any embodiments, the processed mammalian reproductive cells are gathered in a container, tube, or straw. In any embodiments, the mammalian reproductive cells are selected from the group consisting of gametes, haploid cells, germ cells, sex cells, sperm cells, and egg cells. In any embodiments, a sperm cell composition is produced by this processing method.

Processing of raw ejaculate can include many downstream applications. For example, processing of raw ejaculate can include one or more of the applications including, but not limited to sorting, sexing (selecting X-chromosome bearing or Y-chromosome bearing cells), freezing, artificial insemination, and IVF (with and without sexing). In some embodiments, this can include cooling and transport of samples, concentrating sperm cells and suspending before staining/sexing.

One aspect of the disclosure relates to methods of protecting sperm cells throughout the process (e.g., the sexing process or the conventional process), wherein the method includes the step of adding to the sperm cells, an effective amount of a potassium channel blocker compound or a composition thereof. In any embodiments the composition further includes a medium. In any embodiments, the potassium channel blocker compound is included in the medium. In any embodiments, the medium is an extender medium. In any embodiments, the medium is a staining buffer, a cryopreservation buffer, and/or semen processing buffer. In any embodiments, the potassium channel blocker compound is added before, during and/or after the sexing process. In any embodiments, the potassium channel blocker compound is added to the sperm cells and the potassium channel blocker compound has a concentration ranging from 1 μM to 100 μM in the resulting mixture, including, without limitation, about 0.1 μM to about 500 μM, about 0.5 μM to about 250 μM, about 1 μM to about 100 μM, about 2 μM to about 50 μM, about 3 μM to about 30 μM, about 4 μM to about 20 μM, or about 5 μM to about 10 μM.

Another aspect of the disclosure relates to methods of eliciting a positive functional improvement in sperm cells at the post-thaw stage. In any embodiment, the method includes adding to the sperm cells at a pre-freeze, an effective amount of a potassium channel blocker compound or a composition thereof. In any embodiments the composition further includes a medium. In any embodiments, the potassium channel blocker compound is included in the medium. In any embodiments, the medium is an extender medium. In any embodiments, the medium is a staining buffer, a cryopreservation buffer, and/or semen processing buffer. In any embodiments, the potassium channel blocker compound is added before, during and/or after the process (e.g., the sexing process or the conventional process). In any embodiments, the potassium channel blocker compound is added to the sperm cells and the potassium channel blocker compound has a concentration ranging from 1 μM to 100 μM, including, without limitation, about 0.1 μM to about 500 μM, about 0.5 μM to about 250 μM, about 1 μM to about 100 μM, about 2 μM to about 50 μM, about 3 μM to about 30 μM, about 4 μM to about 20 μM, or about 5 μM to about 10 μM.

In any aspects and embodiments, the compositions herein are sperm cell compositions that comprise sperm cells, clofilium tosylate and a medium. The compositions may include seminal fluid components. In some embodiments, the media may include one or more of extender media, staining media (stain TALP), collection media (Part 1 or TRIS A), and cryopreservation media (packaging extender). In some embodiments, the medium may include one or more of NaCl, KCl, Na₂HPO₄, NaHCO₃, MgCl₂.6H₂O (Tyrode's). In some embodiments, the medium may include one or more of Tyrode's, Sodium lactate syrup, Glucose, HEPES, Sodium Pyruvate, BSA (Stain TALP). In some embodiments, the medium may include one or more of Stain TALP, Egg Yolk, Quenching Dye (Red TALP). In some embodiments, the medium may include one or more of Sterile Milli-Q H₂O, TRIS, Egg Yolk, GTLS (Part 1 or TRIS A). In some embodiments, the medium may include one or more of Sterile Milli-Q H₂O, TRIS, Egg Yolk, Glycerol, Green Food Color, GTLS (Part 2 or TRIS B). In some embodiments, the medium may include one or more of TRIS A (Part 1) and TRIS B (Part 2) (Packaging Extender). In some embodiments, the medium may include one or more of Gentamicin Sulfate (powdered, Amresco #0304), Sterile Milli-Q H₂O (Gentamycin Sulfate Solution). In some embodiments, the medium may include one or more of Tylosin (Tylosin Tartrate; Midwest Vet Supply), Sterile Milli-Q H₂O (Tylosin Solution). In some embodiments, the medium may include one or more of Gentamicin Sulfate Solution, Tylosin Solution, Linco-Spectin Stock (50 mg Lincomycin/100 mg Spectinomycin; Midwest Vet Supply) (GTLS).

Other components which may be included in the medium, may include, without limitation, one or more of sodium chloride, potassium chloride, disodium phosphate, sodium bicarbonate, magnesium chloride hexahydrate, D-(+)-Glucose, sodium pyruvate, sodium lactate , HEPES, BSA (Fraction V), red food dye, tris(hydroxymethyl)aminomethane (Trizma), citric acid monohydrate, D-Fructose, egg yolk, glycerol, green food dye, CaCl₂(H₂O)₂. MgCl₂(H₂O)₆, NaHCO₃, NaH₂PO₄ dihydrate, KH₂PO₄, fructose, sorbitol, phosphatidylserine, decursin, zinc chloride, coenzyme Q10, aspirin, linolenic acid, fatty acid supplement, and D-aspartic acid.

In other aspects and embodiments, the present technology includes a container of sperm cells comprising a plurality of sperm cells, a medium, and one or more of potassium channel blocker compound, and combinations thereof. The container may further comprise seminal fluid components. Such containers can be used for storage, or for further procedures such as IVF or AI.

In vitro fertilization can be carried out by methods and procedures known in the art. Many factors can affect successful IVF, including, but not limited to sources of eggs; sperm samples and additional processing/manipulation; fertilization, presence/concentration of media components/sperm cells during fertilization step; and presence/concentration of media components during blastocyst formation/embryo development.

Some aspects and embodiments of the disclosure are illustrated by the following examples. These examples are provided to describe specific embodiments of the technology and do not limit the scope of the disclosure. It will be understood by those skilled in the art that the full scope of the disclosure is defined by the claims appending this specification, and any alterations, modifications, or equivalents of those claims.

### EXAMPLES

### Materials and Methods

Examples of media formulations wherein clofilium tosylate is added are outlined below:
Tyrode's: NaCl, KCl, Na₂HPO₄, NaHCO₃, MgCl₂.6H₂O.
Stain TALP: Tyrode's, Sodium lactate syrup, Glucose, HEPES, Sodium Pyruvate, BSA.
Red TALP: Stain TALP, Egg Yolk, Quenching Dye.
TRIS A (Part 1): Sterile Milli-Q H2O, TRIS, Egg Yolk, GTLS.
TRIS B (Part 2): Sterile Milli-Q H₂O, TRIS, Egg Yolk, Glycerol, Green Food Color, GTLS.
Packaging Extender: TRIS A (Part 1) and TRIS B (Part 2).
Gentamycin Sulfate Solution: Gentamicin Sulfate (powdered, Amresco #0304), Sterile Milli-Q H₂O.
Tylosin Solution: Tylosin (Tylosin Tartrate; Midwest Vet Supply), Sterile Milli-Q H₂O.
GTLS: Gentamicin Sulfate Solution, Tylosin Solution, Linco-Spectin Stock (50 mg Lincomycin/100mg Spectinomycin; Midwest Vet Supply).

IVF Testable Unit Generation Design: This study utilizes sires from two breeds, Holstein and Jersey. The study design includes one ejaculate collection from 10 unique sires to generate sexed semen units in a split batch design with clofilium and a paired control.

Final breed tally of the collections were 7 Holsteins and 3 Jersey sires. Ejaculates selected for use in this trial were processed following standard production procedures outlined in detail below. Insemination doses, freeze canes, and all associated documentation were blind labeled during the incoming quality check. This was to prevent technician bias on quality control assessments after freeze-thaw as well as during the IVF outcome quantitation assessments.

After processing, the sexed semen was packaged and frozen during a regularly scheduled production freeze following standard operating procedures (SOPs). Within one week of unit generation, the outgoing quality control measurements were performed by a trained research technician. Post-thaw motile concentration and the presence/absence of bacterial contamination were completed. Insemination doses had to pass standard production outgoing quality control parameters to be utilized for the IVF trial.

Each facility received the two treatment groups for each individual ejaculate. The two treatments from each split ejaculate were used concurrently to fertilize oocytes from the same pooled batch of slaughterhouse oocytes.

Trained IVF technicians performed the outlined fertilizations. Day 2 cleavage rate, Day 7 and 8 blastocyst rates will be recorded, as well as polyspermy post-fertilization.

Ejaculate Collections: All ejaculate collections were performed on-site by experienced technicians following standard collection procedures.

Ejaculate extension and Incoming Quality Assessment: Ejaculates were transported in an insulated cooler to prevent temperature fluctuation during transport to the second facility. The volume of the ejaculate was determined by mass. Immediately, within 15 minutes, Citrate buffer was added in a 0.5:1 ratio to the ejaculates. GTLS antibiotic solution was added at a 2% v/v of ejaculate.

Within 45 minutes of initial ejaculate collection, the cell concentration and incoming motility parameters were collected. Concentration was determined using a Nucleocounter SP-100™ with Reagent 5100 and SP100 cassettes (ChemoMetec Allerod, Denmark). Motility characteristics were calculated by diluting 10 μL sample in 990 μL motility diluent and reading 7 frames each in 2 chambers of Leja 4 chamber capillary slides with a known chamber depth on a Hamilton Thorne IVOS II using HTCasa II software at 60 Hz frame capture speed in a 37° C. enclosed stage with a Zeiss 10× objective. An ejaculate was utilized if the cell concentration was greater than 500 million/mL, and the percent of progressive motile cells in the sample was ≥65%

Sample Preparation for Sperm Sexing: A stained sample was prepared at room temperature that contained 200 M/mL sperm cells in 0.06 mg/mL Hoechst 33342 diluted to final volume in Stain TALP with Magnetic Beads with (+clofilium) or without (control) such that clofilium would be at a final concentration of 5 μM in the final stained sample volume. The sample was then incubated in a 37° C. water bath for 45 minutes. After 45 minutes Red Stain TALP was added to the stained sample in a 2:1 v/v ratio. The sample+Red TALP was then thoroughly mixed using inversion, filtered using tube top 20 μm Partec filters (Partec#04-0042-2315), and aliquoted into round bottom 5 mL tubes.

Sexing Cytometer Metrics: The stained, filtered sample was then run on proprietary sexing cytometers. The sample throughput was adjusted to 23,000 cells/sec and the detection and kill lasers were focused. To confirm proper laser focus, kill count assessments were performed before collecting sex skewed sample. A successful kill count has a population that is ≥75% dead and ≥95% sliced with at least 200 cells being counted. If an instrument could not achieve the above metrics, the instrument was not used to collect sex skewed semen. After a successful kill count, a gate was placed to collect the X chromosome cells, which is the cell population with the brighter Hoechst 33342 fluorescence as measured with a 355 nm wavelength excitation laser. Cytometer performance metrics were collected 15 minutes after instrument set up, and 15 minutes after the placement of the last sample collection tube, including the height of the Y-peak, the height of the X-peak, the height of the trough from the histogram of events per emitted fluorescent intensity, gated %, and dead %.

Sex Skewed Sample Collection and Processing: The sample was run to collect between 300 and 400 mL of sex skewed sample, the composition of which is approximately 17% TRIS A (Part 1) buffer, 80% sheath fluid, and 2% cell sample. The sample was collected in 50 mL conical tubes containing 5 mLs of TRIS A (Part 1), and each tube was filled to a max volume of 30 mL before being replaced. After the requisite total volume was collected, sexed sperm was centrifuged at room temperature at 2400×g for 10 minutes. The supernatant was aspirated and discarded to reach a 1 mL pellet volume. 144 μL of TRIS A (Part 1)/GTLS mixture containing 40 μM clofilium or no clofilium (control) was added to each pellet after resuspension. The tubes were then placed in beakers filled with 150 mL of room temperature water, to prevent cold shock, and were then transferred to a 4° C. cold room.

After 90 minutes of equilibration at 4° C. the samples were cryoprotected by adding TRIS B (Part 2, which contains glycerol) to a final concentration of 20% v/v of sample in 3 separate additions, 15 minutes apart. The concentration of progressively motile cells was determined using the Hamilton Thorne IVOS II with HTCasa Animal Breeders II software set to the same capture settings listed above, but with a Xenon light source and an Olympus 10× UplanSApo objective. The cryoprotected sample was diluted to final live, motile cell concentration, 2.6 M/mL, in Packaging Extender containing 5 μM clofilium or no clofilium (control) and placed in Mini Straws which hold 0.25 mL volume (IMV technologies, Maple Grove, Minn. USA) using an MX4 straw filling and sealing machine (IMV technologies, Maple Grove, Minn. USA). Filled straws were rapidly cooled using a freeze tunnel before storage in liquid nitrogen.

Outgoing Quality Control Assessment: To assess the number of motile cells that survived the freezing process a single straw is thawed in a 37° C. water bath for 45 seconds. The straw is then plunged into a pre-warmed Eppendorf tube. The sample is then gently vortexed 10 seconds to homogenize the sample. After which 20 μLs of sample is added to 20 μLs of QC diluent, gently vortexed for 5 seconds, and read on the CASA fluorescent settings described above. The remainder of the straw volume is spread on a blood agar plate and left at 37° C. for 24 hours before bacterial colonies are counted.

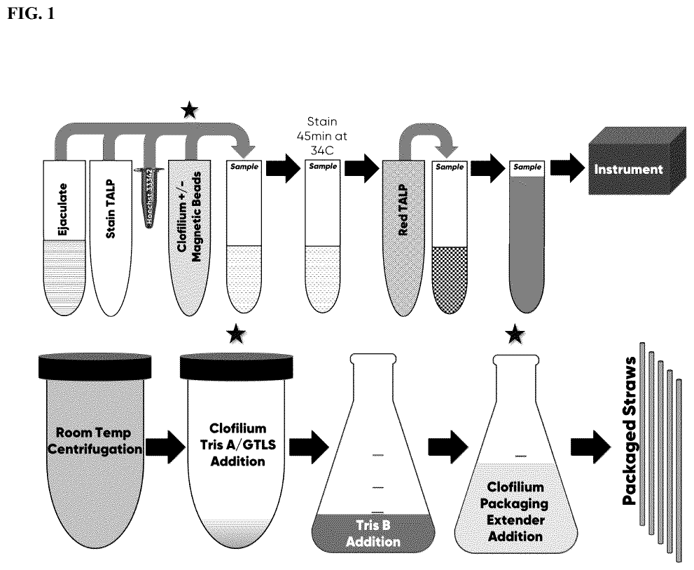

During the course of development of a robust medium for use during various stages of sexing, the inventors tested the effect of clofilium tosylate on the sexing process by adding clofilium tosylate to each media beginning with the staining media, throughout the sexing process, and in the collection and cooling steps and assessed the effects on cell viability and fertility, and impact on production efficiency. Additionally, the addition of clofilium tosylate to samples only after sexing was also tested in order to assess the effects independent of the impacts on processing. Multiple concentrations of clofilium tosylate were tested in various compositions for different applications as well as the effect of timing of addition of clofilium tosylate was studied. The addition of clofilium tosylate was studied at various stages as shown in FIG. 1. For example, clofilium can be first added in a mixture with magnetic beads in the staining step with Stain TALP and Hoechst 33342, added a second time in the Tris A (Part 1 of a two part extender)/GTLS addition after the centrifugation that occurs post-instrument, and added for a third time in the packaging extender pre-freeze. Clofilium is maintained at a steady concentration, (e.g., 5 μM) throughout the process. A citrate-based extender is used pre-instrument and magnetic beads are used to remove dead cells pre-instrument.

### Example 1

Sexing procedures require that spermatozoa survive a multitude of insults, and preliminary data demonstrated that the medium containing clofilium tosylate facilitates successful sexing up to about 24 hours post-collection, depending on the background extender formulation. While scientists have attempted to correlate in vitro sperm characteristics with fertilization outcomes, no such assay has become a gold standard. Cellular assessments of the plasma membrane, intracellular structures, as well as tests of velocity parameters with a CASA system or with the bovine cervical mucus penetration test (BCMPT) often have conflicting results in how they relate to IVF or AI fertility. For example, PI staining of plasma membrane integrity correlating with field fertility for Januskauskas et al. (2003) and no correlation for Oliveira et al. (2012). Also, distance traveled in BCMPT appeared to correlate to NRR (Tas et al., 2007; Bacinoglu et al., 2007) but that this assay did not relate well to IVF outcomes (Keel and Schalue, 2009). These conflicting results prevent relating sperm viability or motility measurements to performance in IVF or AI. Accurately assessing spermatozoa fertility, therefore, requires performing an IVF or AI.

While an AI trial is required to apply the compositions described herein to commercial products, an IVF trial quantifies cleavage and blastocyst conversion rates, providing insight into mechanisms underlying apparent changes in the number of embryos produced (Bermejo-Alvarez et al., 2010; Blondin et al., 2009, Greve and Madison, 1991). Quantified outcomes in this IVF trial will include percent fertilization, cleavage conversion, and blastocyst conversion rates day 7 and day 8. These two time points to quantify blastocyst development are based on literature showing a developmental delay in IVF using sexed semen (Lu et al., 1999), and will indicate whether the compositions described herein change the rate of embryonic development.

Based on the data of velocities measured post freezing after incubation with the compositions described herein and the preliminary IVF assessment with extended, paired ejaculates from two sires, it is hypothesized that extension with the compositions described herein will perform as well as unextended ejaculates. The ability of the extended ejaculates to perform as well as the non-extended ejaculates in IVF would verify that the extender compositions described herein could be used in a sexed semen product and still allow for fertilization and early embryonic development. This increases the time available to sex the ejaculate volume and would allow for an increased number of sexed insemination doses produced per volume of ejaculate collected. This increase in production would improve farmer access to these genetically verified sex-skewed insemination doses.

### Example 2

An illustrative flowchart showing the addition of clofilium tosylate at various stages during the sexing process, is shown in FIG. 1. Each media containing clofilium tosylate is designated by a star. For example, an ejaculate sample is obtained and a volume of Stain TALP and Hoechst 33362 are added such that clofilium tosylate is introduced to the sample at a concentration of 5 uM. The staining step is carried out by incubating the treated ejaculate sample for about 45 minutes at 34° C. Red TALP is added to the stained sample, and the sample is then sorted on a suitable instrument to provide a sex-skewed sample. The sex-skewed sample is subjected to room temperature centrifugation and is then treated with TrisA Part 1 (of a two-part extender)/GTLS, which may include additional clofilium tosylate. Tris B (Part 2) is added to the sample, followed by a packaging extender medium, which may again include clofilium tosylate, and the sample packaged in straws or other packaging for freezing and cryopreservation.

As noted in the foregoing procedure, clofilium tosylate is added at multiple stages (e.g., at the staining step, centrifugation step and prior to cryopreservation step) to maintain the 5 uM concentration. For example, the Stain TALP medium may include 5 uM clofilium or clofilium is added to achieve/maintain 5 uM concentration; TRIS A (Part 1)/GTLS may include 5 uM clofilium or clofilium is added to achieve/maintain 5 uM concentration; and cryopreservation media may include also include 5 uM clofilium or clofilium is added to achieve/maintain 5 uM concentration.

The frozen sample may then be thawed and used for IVF embryo production or artificial insemination. The sample may be checked or evaluated through quality control to determine that fertility/motility levels (see other FIGs) have been achieved.

### Example 3

### In Vitro Fertilization and Assessment

Oocyte prep: Four well fertilization plates are prepared by filling all 4 wells with 400 μL of BO-IVF (MOFA Verona, Wis.) and equilibrated in a 37° C. 5% CO₂ for at least 1 hour. At this same time, four well embryo culture plates filled with 450 μL of BO-IVC (MOFA Verona, Wis.) are made and equilibrated at 37° C. 5% CO₂, 5% O₂. A sample of each lot of BO-IVC used during these fertilizations is aliquoted and stored at −80° C. as control media for assessing conditioned embryo media. All handling of oocytes and zygotes is done with heat pulled glass pipettes.

Cumulus oocyte complexes (COCs) are collected from slaughterhouse ovaries by aspiration. The COCs are kept warm in oocyte maturation media and handled on 37° C. heated stages. COCs are grouped and separated into 3 wells of 60 oocytes each per treatment group in a 4 well plate.

Semen prep: Three insemination straws per treatment group are thawed at 37° C. for 45 seconds. They are then layered over 80% BoviPure™ density gradient (Nidacon international AB. Sweden). The samples are centrifuged at 500×g for 15 minutes, aspirated close to the pellet, and then resuspended in warm TL HEPES (MOFA Verona, Wis.). They are centrifuged at 300×g for 5 minutes, aspirated to 100 μLs, and the pellet is resuspended in that low volume. A 5 μL sample aliquot is added to 95 μLs 4% NaCl to immobilize the cells, and cell concentration is quantified using a hemocytometer. Cells with visible membrane damage are not counted towards cell density calculations. Sperm suspension is added to the COC containing wells at 1.2 million sperm per well (20,000 sperm/oocyte).

Cumulus oocyte complex removal: 24 hours after sperm addition, COCs from the same treatment group are pooled in a 15-mL conical tube containing 0.5 mL TL HEPES with 1 mg/mL hyaluronidase. COCs are vortexed for 1 minute, put back into the 37° C. heating block for 1 minute, and vortexed again for 1 minute. The presumptive zygotes are washed in TL HEPES plates and then placed in the embryo culture plates containing maturation media that are equilibrated for 24 hours prior to use (oocyte prep above). The presumptive zygote containing plates are then placed at 37° C. 5% CO₂, 5% O₂ for the rest of the IVF trial.

Development assessments: Developmental assessments are performed three times during the 8-day post-fertilization incubation. Cleavage events are quantified 48 hours after initial fertilization. Blastocysts are scored on a binary scale of yes/no blastocyst based on its developmental stage. If the embryo had reached at least the early blastocyst stage it is scored as a blastocyst. The differences between early, expanding, and hatched blastocysts are not recorded, nor are the blastocysts scored, but blastocysts are fixed to facilitate future characterization. Blastocyst conversion per oocyte is visually determined on both day 7 and day 8 after initial fertilization. All determinations of developmental stages are done by trained IVF technicians using a dissecting scope on a heated stage set to 37° C.

Assessment of early fertilization events: 24 hours after initial fertilization and after the presumptive zygotes are stripped of their COCs, a subset of 20-30 zygotes are fixed and stained using a proprietary kit created to assess for monospermic/polyspermic events. The DNA stain is Hoechst 33342. All presumptive zygotes are scored in 1 of 4 categories: monospermic fertilization, polyspermic fertilization, unfertilized, or other. The other category encompasses zygotes that are present, but un-scorable due to either obscuring fluorescence from COC not fully removed or because the zygote is fragmented. Those zygotes presenting with two pronuclei are considered monospermic, and those presenting with 3 or more are scored as polyspermic (Yang et al., 1993).

Statistical Analysis: All statistical analysis is performed using OriginPro 2021 64-bit software. Threshold for significance is set at α=0.05.

### Results

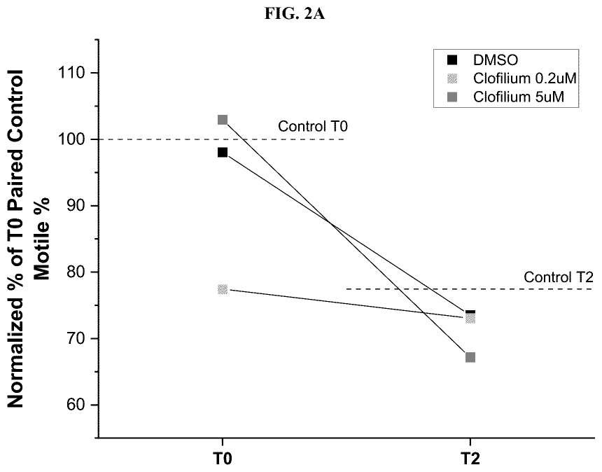

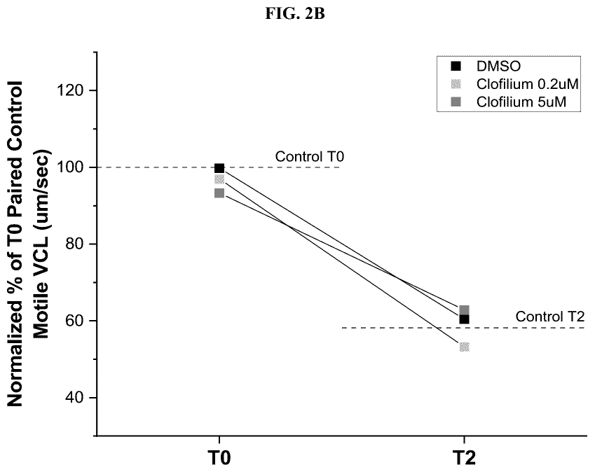

FIGS. 2A and 2B show a comparison in percentage motility and curvilinear velocity (VCL), respectively, after 2 hr storage of post-thaw straw material between Clofilium tosylate (0.2 μM and 5 μM), DMSO and a Control. The screening was performed using DMSO control (match highest DMSO concentration) and peroxidase (POD) SOP control with post-thaw straw material to find any toxicities (test T0 and T2 post thaw). VCL and Motile % were compared to averaged POD and DMSO control. Loss of >10% Motile M/mL compared to time matched POD SOP control at either/or both time points was considered to be indicative of toxic effect on cells.

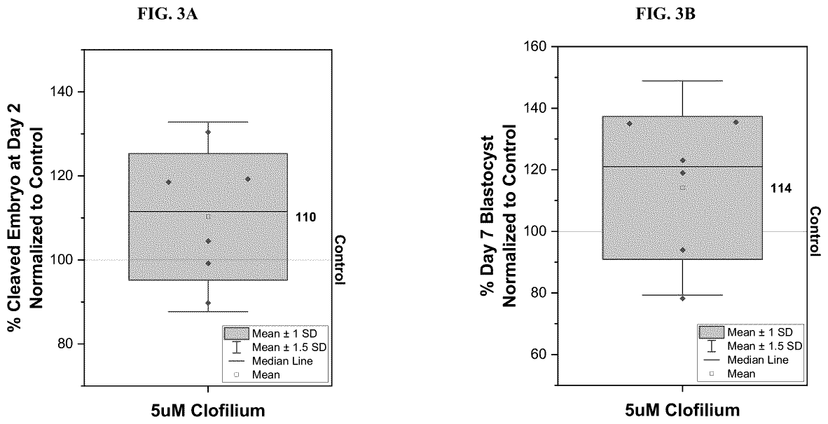

Clofilium demonstrated potential for IVF gains in preliminary trials. Sexcel straws from N=2 sires produced by adding 5 μM Clofilium tosylate throughout the sexing process. IVF was performed in triplicates with n=100 oocytes/treatment/replicate in Genus R&D IVF media. FIGS. 3A and 3B show an improvement (normalized to the control process) in percentage of normalized cleaved embryos at day 2 and in percentage of day 7 blastocysts.

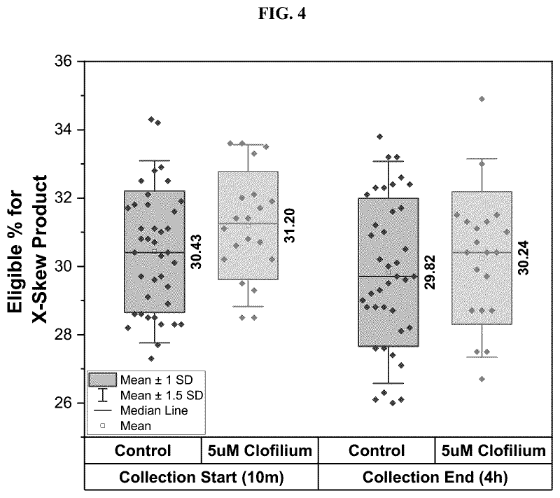

Clofilium maintained improved % eligible for sexing duration (n=10 sires). FIG. 4 shows an improvement in the percentage of eligible cells (live, sexed X chromosome bearing cells) compared to SOP at the start of the collection process and at the end of the collection process. 5 μM Clofilium tosylate increased eligible percent, suggesting increased straw output. Skew (percentage of X-chromosome bearing sperm cells in this example) remains unaffected by the use of clofilium.

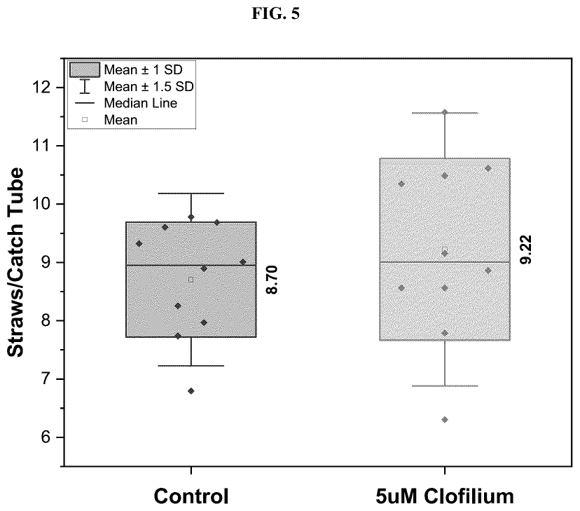

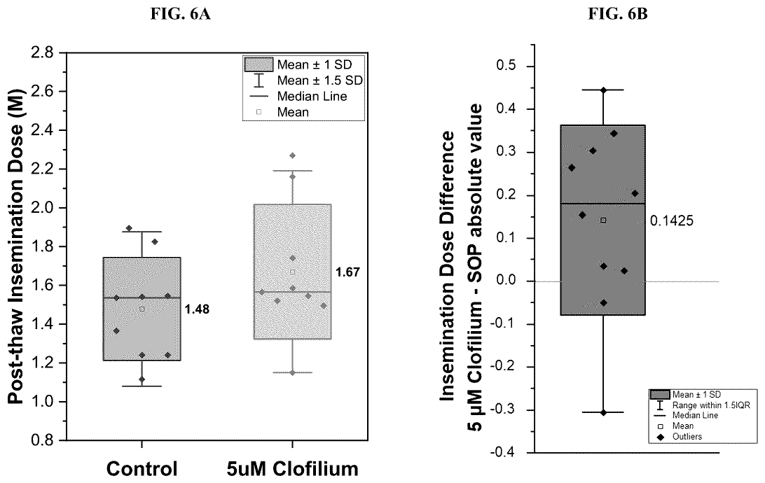

Clofilium may also improve motile cell yield (n=10 sires). FIGS. 5, 6A and 6B show an improvement in the number of straws that may be obtained from a single sample catch tube (post instrument processing). There are also more motile cells (in millions) per insemination dose with clofilium, both in number and in absolute value compared to SOP.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims.

### First Set of Lettered Paragraphs

A. A composition comprising:
   a reproductive cell selected from the group consisting of a sperm cell, an oocyte, an embryo, an embryonic stem cell and a spermatogonial stem cell;
   an effective amount of a potassium channel blocker compound for enhancing cell viability during and/or after one or more of staining, freezing, thawing, cell sample enrichment, packaging, or in vitro fertilization; and
   a medium.
B. The composition of Paragraph A, wherein the potassium channel blocker compound is clofilium tosylate.
C. The composition of Paragraphs A or B, wherein the potassium channel blocker compound is present at a concentration ranging from about 1 nM to 100 mM.
D. The composition of any one of Paragraphs A-C, wherein the composition comprises a plurality of sperm cells.
E. The composition of any one of Paragraphs A-D, wherein the sperm cells comprises mammalian sperm cells.
F. The composition of Paragraph E, wherein the mammalian sperm cells comprises human, bovine, porcine, equine, ovine, elk, or bison sperm cells.
G. The composition of any one of Paragraphs A-F, wherein the medium comprises one or more of a cryoprotectant, a buffer, a diluent, an energy source, an extender medium, an antibiotic, and a bolus.
H. A media composition comprising an amount of a potassium channel blocker compound effective to improve the function of sperm, oocyte, embryo, embryonic stem cell or spermatogonial stem cell, wherein the improvement in function comprises improvement in sexed semen production, improvement in efficiency of the sexing process, improvement in fertility/viability of sexed semen, improvement in in vitro fertilization, improvement in rates of embryo production and/or increased implantation, and live births.
I. A composition comprising semen, an extender composition, and an effective amount of a potassium channel blocker compound, wherein the semen provides a concentration of motile sperm in the composition ranging from 0.01 M motile sperm/ml to 2000 M motile sperm/ml.
J. A method for enhancing reproductive cell viability during and/or after one or more of staining, freezing, thawing, cell sample enrichment, packaging, or in vitro fertilization, the method comprising, adding to the reproductive cells, a composition comprising an effective amount of a potassium channel blocker compound and a medium, wherein the potassium channel blocker compound is added at a concentration ranging from 1 nM to 100 mM.
K. A method of protecting sperm cells throughout the sexing process comprising adding to the sperm cells a composition comprising an effective amount of a potassium channel blocker compound and a medium, wherein the potassium channel blocker compound is added before, during and/or after the sexing process at a concentration ranging from 1 nM to 100 mM.
L. A method of eliciting a positive functional improvement in sperm cells at the post-thaw stage, the method comprising, adding to the sperm cells at a pre-freeze, a composition comprising an effective amount of a potassium channel blocker compound and a medium, wherein the potassium channel blocker compound is added at a concentration ranging from 1 nM to 100 mM.
M. A method of improving quality of a semen sample comprising contacting the semen sample with a composition comprising an effective amount of a potassium channel blocker compound and a medium, wherein the potassium channel blocker compound is added at a concentration ranging from 1 nM to 100 mM.
N. The method of any one of Paragraphs J-M, wherein the potassium channel blocker compound is clofilium tosylate.
O. The method of any one of Paragraphs J-N, wherein the reproductive cell is selected from the group consisting of a sperm cell, an oocyte, an embryo, an embryonic stem cell and a spermatogonial stem cell.
P. The method of Paragraph O, wherein the reproductive cells comprise sperm cells.
Q. The method of Paragraph P, wherein the sperm cells comprises mammalian sperm cells.
R. The method of Paragraph Q, wherein the mammalian sperm cells comprises human, bovine, porcine, equine, ovine, elk, or bison sperm cells.
S. The method of any one of Paragraphs J-R, wherein the medium comprises one or more of a cryoprotectant, a buffer, a diluent, an energy source, an extender medium, an antibiotic, and a bolus.
T. The method of any one of Paragraphs J-S, wherein the medium comprises an extender medium.
U. The method of any one of Paragraphs J-T, further comprising adding the potassium channel blocker to the reproductive cells or sperm cells two or more times at different points in the method to maintain an effective amount of the potassium channel blocker.
V. The method of any one of Paragraphs J-U, further comprising adding the potassium channel blocker to the reproductive cells or sperm cells at the staining step.
W. The method of any one of Paragraphs J-V, further comprising adding the potassium channel blocker to the reproductive cells or sperm cells at the centrifugation step.
X. The method of any one of Paragraphs J-W, further comprising adding the potassium channel blocker to the reproductive cells or sperm cells prior to the cryopreservation step.

### Second Set of Lettered Paragraphs

A. A composition comprising:
   a reproductive cell selected from the group consisting of a sperm cell, an oocyte, an embryo, an embryonic stem cell and a spermatogonial stem cell; and
   an effective amount of a potassium channel blocker compound for enhancing cell viability during and/or after one or more of storage, staining, freezing, thawing, cell sample enrichment, packaging, or in vitro fertilization.
B. The composition of Paragraph A, wherein the potassium channel blocker compound is clofilium tosylate.
C. The composition of Paragraph A or Paragraph B, wherein the potassium channel blocker compound is present at a concentration ranging from about 1 nM to 100 mM.
D. The composition of any one of Paragraphs A-C, wherein the composition comprises a plurality of sperm cells.
E. The composition of any one of Paragraphs A-D, wherein the sperm cells comprises mammalian sperm cells.
F. The composition of Paragraph E, wherein the mammalian sperm cells comprises human, bovine, porcine, equine, ovine, elk, or bison sperm cells.
G. The composition of any one of Paragraphs A-F, further comprising a medium.
H. The composition of Paragraph G, wherein the medium comprises one or more of a cryoprotectant, a buffer, a diluent, an energy source, an extender medium, and an antibiotic.
I. A media composition comprising an amount of a potassium channel blocker compound effective to improve the function of sperm, oocyte, embryo, embryonic stem cell or spermatogonial stem cell, wherein the improvement in function comprises improvement in one or more of sexed semen production, efficiency of the sexing process, fertility/viability/physiological function of sexed semen, in vitro fertilization, rates of embryo production and/or increased implantation, and live births.
J. A composition comprising semen, an extender composition, and an effective amount of a potassium channel blocker compound, wherein the semen provides a concentration of motile sperm in the composition ranging from 0.01 M motile sperm/ml to 2000 M motile sperm/ml.
K. A method for enhancing reproductive cell viability during and/or after one or more of staining, freezing, thawing, cell sample enrichment, packaging, or in vitro fertilization, the method comprising, adding to the reproductive cells, an effective amount of a potassium channel blocker compound or a composition thereof, wherein the potassium channel blocker compound or a composition thereof is added to result in a composition comprising from 1 nM to 100 mM of potassium channel blocker compound.
L. A method of protecting sperm cells throughout the sexing process comprising adding to the sperm cells an effective amount of a potassium channel blocker compound or a composition thereof, wherein the potassium channel blocker compound or a composition thereof is added before, during and/or after the sexing process to result in a composition comprising from 1 nM to 100 mM of potassium channel blocker compound.
M. A method of eliciting a positive functional improvement in sperm cells at the post-thaw stage, the method comprising, adding to the sperm cells at a pre-freeze, an effective amount of a potassium channel blocker compound or a composition thereof, wherein the potassium channel blocker compound or a composition thereof is added to result in a composition comprising from 1 nM to 100 mM of potassium channel blocker compound.
N. A method of improving quality of a semen sample comprising contacting the semen sample with an effective amount of a potassium channel blocker compound or a composition thereof, wherein the potassium channel blocker compound or a composition thereof is added to result in a composition comprising from 1 nM to 100 mM of potassium channel blocker compound.
O. The method of any one of Paragraphs K-N, wherein the potassium channel blocker compound is clofilium tosylate.
P. The method of Paragraph K, wherein the reproductive cell is selected from the group consisting of a sperm cell, an oocyte, an embryo, an embryonic stem cell and a spermatogonial stem cell.
Q. The method of Paragraph K or P, wherein the reproductive cells comprise sperm cells.
R. The method of Paragraph N, wherein the semen sample comprises sperm cells.
S. The method of any one of Paragraphs K-R, wherein the sperm cells comprises mammalian sperm cells.
T. The method of Paragraph S, wherein the mammalian sperm cells comprises human, bovine, porcine, equine, ovine, elk, or bison sperm cells.
U. The method of any one of Paragraphs K-T, wherein the potassium channel blocker compound is included in a medium.
V. The method of Paragraph U, wherein the medium comprises one or more of a cryoprotectant, a buffer, a diluent, an energy source, an extender medium, and an antibiotic.
W. The method of Paragraph U or Paragraph V, wherein the medium comprises an extender medium.
X. The method of any one of Paragraphs K-W, further comprising adding the potassium channel blocker to the reproductive cells or sperm cells one or more times at different points in the method to maintain an effective amount of the potassium channel blocker.
Y. The method of any one of Paragraphs K-X, further comprising adding the potassium channel blocker to the reproductive cells or sperm cells at the staining step.
Z. The method of any one of Paragraphs K-Y, further comprising adding the potassium channel blocker to the reproductive cells or sperm cells at the centrifugation step.
AA. The method of any one of Paragraphs K-Z, further comprising adding the potassium channel blocker to the reproductive cells or sperm cells prior to the cryopreservation step.

What is claimed is:

1. A composition comprising:
a reproductive cell selected from the group consisting of a sperm cell, an oocyte, an embryo, an embryonic stem cell and a spermatogonial stem cell; and
an effective amount of a potassium channel blocker compound for enhancing cell viability during and/or after one or more of storage, staining, freezing, thawing, cell sample enrichment, packaging, or in vitro fertilization.

2. The composition of claim 1, wherein the potassium channel blocker compound is clofilium tosylate.

3. The composition of claim 1, wherein the potassium channel blocker compound is present at a concentration ranging from about 1 nM to 100 mM.

4. The composition of claim 1, wherein the composition comprises a plurality of sperm cells.

5. The composition of claim 1, wherein the sperm cells comprises mammalian sperm cells.

6. The composition of claim 5, wherein the mammalian sperm cells comprises human, bovine, porcine, equine, ovine, elk, or bison sperm cells.

7. The composition of claim 1, further comprising a medium.

8. The composition of claim 7, wherein the medium comprises one or more of a cryoprotectant, a buffer, a diluent, an energy source, an extender medium, and an antibiotic.

9. A media composition comprising an amount of a potassium channel blocker compound effective to improve the function of sperm, oocyte, embryo, embryonic stem cell or spermatogonial stem cell, wherein the improvement in function comprises improvement in one or more of sexed semen production, efficiency of the sexing process, fertility/viability/physiological function of sexed semen, in vitro fertilization, rates of embryo production and/or increased implantation, and live births.

10. The media composition of claim 9, comprising semen, an extender composition, and an effective amount of a potassium channel blocker compound, wherein the semen provides a concentration of motile sperm in the composition ranging from 0.01 M motile sperm/ml to 2000 M motile sperm/ml.

11. A method for enhancing reproductive cell viability during and/or after one or more of staining, freezing, thawing, cell sample enrichment, packaging, or in vitro fertilization, the method comprising, adding to the reproductive cells, an effective amount of a potassium channel blocker compound or a composition thereof to, wherein the potassium channel blocker compound or a composition thereof is added to result in a mixture comprising from 1 nM to 100 mM of the potassium channel blocker compound.

12. The method of claim 11, wherein the potassium channel blocker compound is clofilium tosylate.

13. The method of claim 11, wherein the reproductive cell is selected from the group consisting of a sperm cell, an oocyte, an embryo, an embryonic stem cell and a spermatogonial stem cell.

14. The method of claim 11, wherein the reproductive cells comprise sperm cells.

15. The method of claim 11, wherein the cell sample is semen sample comprising sperm cells.

16. The method of claim 11, wherein the sperm cells comprises mammalian sperm cells.

17. The method of claim 16, wherein the mammalian sperm cells comprises human, bovine, porcine, equine, ovine, elk, or bison sperm cells.

18. The method of claim 11, wherein the potassium channel blocker compound is included in a medium.

19. The method of claim 18, wherein the medium comprises one or more of a cryoprotectant, a buffer, a diluent, an energy source, an extender medium, and an antibiotic.

20. The method of claim 18, wherein the medium comprises an extender medium.

21. The method of claim 11, further comprising adding the potassium channel blocker to the reproductive cells or sperm cells one or more times at different points in the method to maintain an effective amount of the potassium channel blocker.

22. The method of claim 11, further comprising adding the potassium channel blocker to the reproductive cells or sperm cells at the staining step.

23. The method of claim 11, further comprising adding the potassium channel blocker to the reproductive cells or sperm cells at the centrifugation step.

24. The method of claim 11, further comprising adding the potassium channel blocker to the reproductive cells or sperm cells prior to the cryopreservation step.
